(19) United States
(12) Patent Application Publication
(10) Pub. No.: US 2024/0274228 A1
(43) Pub. Date: Aug. 15, 2024

(54) METHOD FOR PREDICTING AND EVALUATING FUNCTION OF BIOMATERIAL

(71) Applicant: PEKING UNIVERSITY SCHOOL AND HOSPITAL OF STOMATOLOGY, Beijing (CN)

(72) Inventors: Xuliang DENG, Beijing City (CN); Xuehui ZHANG, Beijing City (CN); Yingying ZHOU, Beijing City (CN); Xianfeng PING, Beijing City (CN)

(21) Appl. No.: 18/429,680

(22) Filed: Feb. 1, 2024

Related U.S. Application Data
(63) Continuation of application No. PCT/CN2021/119233, filed on Sep. 18, 2021.

(30) Foreign Application Priority Data
Aug. 3, 2021 (CN) 202110884816.5

(51) Int. Cl. G16B 20/00; C12Q 1/6874; C12Q 1/6881; G06N 5/022; G16B 40/20

(52) U.S. Cl. CPC G16B 20/00; C12Q 1/6874; C12Q 1/6881; G06N 5/022; G16B 40/20; C12Q 2600/158

(57) ABSTRACT

A method for predicting and evaluating the function of a biomaterial includes (1) in the environment of a material to be tested, culturing human bone marrow mesenchymal stem cells; (2) collecting the human bone marrow mesenchymal stem cells cultured in step (1), extracting total RNA, performing purification, building a library, and sequencing a transcriptome to obtain transcriptome data of samples to be tested; and (3) subjecting the transcriptome data of the samples to be tested obtained in the step (2) to batch effect correction and feature extraction, and then inputting the resulting data to a function prediction and evaluation model of the present invention, and calculating the samples to be tested as confidence coefficients of different cell types respectively. The present invention can be used in the field of biomaterial function prediction and evaluation.

### CROSS REFERENCE TO RELATED APPLICATIONS

This application is a Continuation of International Patent Application No. PCT/CN2021/119233, filed Sep. 18, 2021, which claims priority to Application No. CN202110884816.5, entitled “METHOD FOR PREDICTING AND EVALUATING FUNCTION OF BIOMATERIAL”, filed on Aug. 3, 2021, the contents of which are incorporated herein by reference in their entireties.

### TECHNICAL FIELD

The present invention relates to an evaluation model of a biomaterial, and in particular to a method for predicting and evaluating the function of a biomaterial.

### BACKGROUND ART

At present, medical materials are mainly evaluated in terms of physicochemical and biological properties at home and abroad. The evaluation of biological properties focuses on the evaluation of biological toxicity and safety, but there is no unified evaluation system for functional evaluation. For example, the evaluation of the function of regulating the stem cell fate of biological materials has not been included in the national evaluation standards for effectiveness and safety of medical biomaterials. Therefore, the material evaluation data in this field are generated in various biomaterial research laboratories, and the sample database is heterogeneous due to the lack of uniform standards for characterization methods and techniques, etc. In addition, most current functional evaluation experiments are limited to a single index. The identity of a cell is reflected in the expression of its specific genes, and thus the current identification of cell classes is often the identification of the expression of a single specific gene. For example, genes BMP2, Runx2, COL1, etc., which are highly expressed in osteoblasts, are detected via quantitative real-time polymerase chain reaction (qPCR) at the gene level, or osteocalcin (OCN) and bone alkaline phosphatase (ALP) are detected via western blot (WB) at the protein level.

However, the use of traditional single-index evaluation methods has great limitations, which are mainly reflected in the following aspects: (1) The detection of a single gene via qPCR is not enough to accurately determine the identity of a cell, because the same gene may be highly expressed in a variety of cell classes; and in addition, even if only a part of the cells highly express the gene, it may still lead to the detection of overall high expression via qPCR. (2) In order to improve the accuracy, multiple genes often need to be detected via qPCR, resulting in a waste of labor. (3) It is difficult to compare the evaluations of different materials: evaluations based on different indices cannot be directly compared, and even if the indices are the same, it is still difficult to compare the evaluations due to the lack of standard quantification. (4) It is impossible to provide a complete picture of the state of cell differentiation, neither the proportion of differentiated cells nor whether the cells have differentiated in the direction of osteocytes.

In summary, the effect of the expression of a single biomarker molecule on the evaluation of the direction of cell differentiation is not quantifiable, the lack of quantifiable evaluation of the complete picture of cell differentiation makes the functional design and optimization of novel biomaterials lack of theoretical and data support, it is difficult to optimize the physicochemical parameters of the material systems by high-throughput screening, and the biological properties of the novel biomaterials are also unpredictable.

### SUMMARY OF THE INVENTION

The present invention provides a highly accurate and predictable method for predicting and evaluating the function of a biomaterial, in order to solve the technical problems of labor intensiveness, a long experiment period and large sample heterogeneity in the existing evaluation methods.

To this end, the present invention provides a method for predicting and evaluating the function of a biomaterial, comprising the following steps: (1) in the environment of a material to be tested, culturing human bone marrow mesenchymal stem cells; (2) collecting the human bone marrow mesenchymal stem cells cultured in step (1), extracting total RNA from the cells, and subjecting the total RNA to purification, library construction and transcriptome sequencing to obtain transcriptome data of samples to be tested; and (3) subjecting the transcriptome data of the samples to be tested obtained in step (2) to batch effect adjustment and feature extraction, then inputting the resulting data to a function prediction and evaluation model of the present invention, and calculating the confidence coefficients of the samples to be tested being different cell classes, respectively.

Preferably, a method for constructing the function prediction and evaluation model in step (3) comprises the following steps: (a) dividing transcriptome data collected from the public database GEO into training sets and testing sets, and subjecting the training sets and the testing sets to batch effect adjustment, respectively; (b) extracting the gene expression profiles of four cell classes based on data of the training sets, and subjecting the transcriptome data to feature extraction; (c) training and optimizing machine learning models based on the data of the training sets, and obtaining an MeD-P intelligent prediction model after integration; and (d) inputting data of the testing sets to the MeD-P intelligent prediction model to obtain predicted cell classes of samples in the testing sets, comparing the predicted cell classes with true cell classes of the samples, and statistically calculating the indices of accuracy, precision, recall and F1-score of the model.

Preferably, in step (a), the batch effect adjustment is integrated optimization based on the ComBatseq algorithm and the DaMiRseq algorithm; the classes and batches of samples in the training sets are known; and the classes of the samples in the testing sets are unknown, the batch effect adjustment of the testing sets is based on parameters generated by the batch effect adjustment of the training sets, and each testing set is adjusted independently.

Preferably, in step (b), the feature extraction is integrated extraction based on the DaMiRseq algorithm and the DESeq2 algorithm; after subjecting the training sets to batch effect adjustment, specifically expressed genes of the four cell classes are extracted according to the classes of the samples; and expression matrices of the feature genes are extracted from the data of the training sets and the testing sets after batch effect adjustment, respectively.

Preferably, in step (c), the model is first trained and optimized on the training sets, and then the evaluation indices of the model are calculated on the testing sets, and the MeD-P constructed comprises nine machine learning algorithm of SVM-R, SVM-L, RF, GNB, LDA, LR, MLP, RidgeCV and kNN.

The present invention has the following beneficial effects:

The present invention designs and constructs a method for predicting and evaluating the function of a biomaterial based on transcriptome for quantitative evaluation, in which the gene expression profile of the cell transcriptome to be tested is compared with the gene expression profiles of different cell classes differentiated from stem cells constructed in advance, in order to obtain a complete picture of the state of cell differentiation induced by a biomaterial.

Specifically, the present invention trains an intelligent prediction model that can distinguish samples of four cell classes of osteoblasts, chondrocytes, adipocytes and undifferentiated mesenchymal stem cells by integrating nine machine learning algorithms of SVM-R, SVM-L, RF, GNB, LDA, LR, MLP, RidgeCV and kNN. Compared with the traditional biomarker-dependent evaluation methods, the accuracies in determining the four cell classes are significantly improved. Moreover, the present invention utilizes the RNAseq data derived from public databases of human bone marrow mesenchymal stem cells before and after chemical induction and culture with biological materials as test samples, and inputs the RNAseq data to a prediction model constructed based on a database of the gene expression profiles of reference samples. The results show that the cell classes predicted by the intelligent model are consistent with the phenotypes of the test samples.

### BRIEF DESCRIPTION OF THE DRAWINGS

FIG. 1 illustrates the hierarchical clustering diagram of the RNAseq data derived from the public databases in the present invention, in which the abnormal samples above the horizontal line are removed by means of the correlation coefficients among the samples, and the remaining samples are used for the construction of the database of the gene expression profiles of the reference samples.

FIGS. 2A-2D illustrate the quantitative histograms of the percentages of variance explained of the database of the gene expression profiles of the reference samples and the gene expression boxplots of the reference samples before and after batch effect adjustment in the present invention, in which FIG. 2A shows that before batch effect adjustment, the percentages of variance explained by batches in the reference database are significantly higher than those explained by cell classes, indicating that the differences among the samples are mainly due to batch effect; FIG. 2B shows that before batch effect adjustment, the gene expression distributions of the samples in the reference database are inconsistent among batches, and there is obvious batch effect; FIG. 2C shows that after batch effect adjustment, the percentages of variance explained by cell classes in the reference database are significantly increased and higher than those explained by batch effect; and FIG. 2D shows that after batch effect adjustment, the gene expression distributions of the samples in the reference database tend to be consistent among batches, and the batch effect is significantly adjusted.

FIG. 3A and FIG. 3B illustrate the visualization diagrams of the samples in the reference database by tSNE dimension reduction before and after data preprocessing in the present invention, in which FIG. 3A shows that before data preprocessing, the samples are clustered by batches after dimension reduction; and FIG. 3B shows that after two preprocessing steps of batch effect adjustment and feature extraction, the samples are clustered by cell classes after dimension reduction, and the samples of the same cell class will be clustered together in big data visualization.

FIG. 4 illustrates the gene expression heat map of samples of four cell classes of osteoblasts, chondrocytes, adipocytes and undifferentiated mesenchymal stem cells after feature extraction in the present invention, showing that after the gene expression profiles of the feature genes are extracted, the four cell classes of osteoblasts, chondrocytes, adipocytes and undifferentiated mesenchymal stem cells are obviously different, in which the ordinate is the gene name, and the abscissa is the sample.

FIG. 5A and FIG. 5B illustrate the accuracy comparison of the classic machine learning models in the present invention in predicting the sample cell class and the receiver operating characteristic curves of the optimized intelligent prediction model, in which FIG. 5A shows that when evaluating the accuracies of the models in predicting the tri-lineage differentiation of mesenchymal stem cells on samples in the testing sets, the overall accuracies of the SVM-R, SVM-L, GNB, LR and kNN models in predicting samples of four cell classes are all higher than 90%, among which the kNN model, the default setting of MeD-P, shows the highest prediction accuracy of up to 90.63%; and FIG. 5B shows the receiver operating characteristic curves (ROC curves) of the MeD-P intelligent prediction model selecting the kNN model as the default setting, in which the ordinate is the true positive rate, and the abscissa is the false positive rate, and the average receiver operating characteristic curve is close to the upper left corner, and the area under the curve (AUC value) is 0.966, close to 1, indicating that the prediction model has excellent classification performance.

FIG. 6 illustrates the evaluation report of the classification performance of the optimized intelligent prediction model in the present invention, in which the RNAseq data derived from the public databases of human bone marrow mesenchymal stem cells before and after chemical induction to three directions of osteogenesis, chondrogenesis and adipogenesis are input to the intelligent prediction model as test samples, and the predicted cell class of each sample is obtained after calculation, so as to evaluate the classification performance of the intelligent prediction model, and it can be seen that the test samples of the four classes can all obtain a high F1-score, indicating the two indices of overall precision and overall recall, suggesting that the intelligent prediction model has excellent performance in classifying samples of four cell classes of osteoblasts, chondrocytes, adipocytes and undifferentiated mesenchymal stem cells.

FIG. 7 illustrates the flow chart of the method for constructing the function prediction and evaluation model in the present invention.

FIGS. 8A-8D shows that machine learning performs well in evaluating the hMSCs lineage fate. FIG. 8A The line chart shows that compared with the random 343 genes and the traditional marker genes, the reference feature genes obtain higher per-class accuracy and per-class specificity, which are slightly higher than those of the all-gene set. FIG. 8B The line chart shows that compared with the random 343 genes, the traditional marker genes and the all-gene set, the reference feature genes obtain higher per-class precision and per-class recall. FIG. 8C The line chart shows that compared with the random 343 genes, the traditional marker genes and the all-gene set, the reference feature genes obtain the highest per-class F1-score. FIG. 8D The line chart shows that compared with the random 343 genes, the traditional marker genes and the all-gene set, the reference feature genes obtain the highest overall accuracy and overall specificity.

FIGS. 9A-9B-2 shows the performance comparison of kNN models established based on the reference feature genes and the traditional marker genes in the sample testing sets on Day 7 of culture. FIG. 9A The overall accuracies of the kNN models established based on the reference feature genes and the traditional marker genes in the sample testing sets on Day 7. FIGS. 9B-1 and 9B-2 The reports on the classification prediction errors show the numbers of samples predicted correctly and incorrectly in the sample testing sets on Day 7 by the kNN models established using the traditional marker genes FIG. 9B-1 and the reference feature genes FIG. 9B-2, respectively.

FIGS. 10A-10J-2 shows that MeD-P predict the representative biomaterial-induced hMSCs lineage fate accurately. FIG. 10A The flow chart of using the MeD-P model to evaluate the regenerative potential of representative biomaterials. Briefly, hBM-MSCs are cultured on biomaterials, and harvested after 7 days of culture for RNA-seq. The processed RNA-seq data are input to the model to generate a report predicting the tri-lineage differentiation probabilities. (FIG. 10B, FIG. 10C) The reports on the differentiation probabilities derived from MeD-P predict the osteo-inductive function of the 3D-printed β-TCP scaffolds. FIG. 10D The representative Alizarin Red S (ARS) staining microscopy image of hBM-MSCs after 21 days of culture on the β-TCP scaffolds. (FIG. 10E, FIG. 10F) The reports on the differentiation probabilities derived from MeD-P predict the adipo-inductive and osteo-inductive functions of the PLLA nanofibrous membranes in the AL and RD groups, respectively. FIG. 10G-1 and FIG. 10G-2 The representative FIG. 10G-1 ARS staining microscopy images and FIG. 10G-2 relative ARS quantification of hBM-MSCs after 21 days of culture on the PLLA nanofibrous membranes. (FIG. 10H, FIG. 10I) The reports on the differentiation probabilities derived from MeD-P show that the sandblasting and acid-etching (SLA)-treated Ti-6Al-4V alloy substrates have strong osteo-inductive function. FIG. 10J-1 and FIG. 10J-2 The representative FIG. 10J-1 immunofluorescence microscopy images and FIG. 10J-2 mean fluorescence intensities of BMP2 protein expression in hBM-MSCs after 3 days of culture on the Ti-6Al-4V alloy substrates. Error bars represent standard error of mean, n=3. ** p<0.01, indicating a statistically significant difference.

FIGS. 11A and 11B shows FIG. 11A the mean differentiation index scores of hBM-MSCs before and after 7 days of culture on the 3D-printed β-TCP scaffolds. FIG. 11B The heat map shows that the expression of the osteogenic marker genes in hBM-MSCs after 7 days of culture on the 3D-printed β-TCP scaffolds is upregulated, which is similar to what is observed in hBM-MSCs cultured in an osteo-inductive medium. Error bars represent standard error of mean, n=3.

FIGS. 12A and 12B shows FIG. 12A the mean differentiation index scores of hBM-MSCs after 7 days of culture on the PLLA nanofibrous membranes in the AL and RD groups. FIG. 12B The heat map shows that the expression of the osteogenic marker genes in hBM-MSCs after 7 days of culture on the randomly-oriented spun PLLA nanofibrous membranes is upregulated, and the expression of the adipogenic marker genes in hBM-MSCs after 7 days of culture on the aligned spun PLLA nanofibrous membranes is upregulated. Error bars represent standard error of mean, n=3.

FIGS. 13A and 13B shows FIG. 13A the mean differentiation index scores of hBM-MSCs before and after 7 days of culture on the sandblasted and acid-etched Ti-6Al-4V alloy substrates. FIG. 13B The heat map shows that the expression of the osteogenic marker genes in hBM-MSCs after 7 days of culture on the sandblasted and acid-etched Ti-6Al-4V alloy substrates is upregulated.

FIGS. 14A-14G-2 shows that MeD-P can predict the mesenchymal stem cell lineage fate regulated by electroactivity and chirality accurately. (FIG. 14A, FIG. 14B) The reports on the differentiation probabilities derived from MeD-P predict the osteo-inductive function of the BTO NPs/P(VDF-TrFE) membranes in the unpolarized (UP) and polarized (P) groups. FIGS. 14C-1 and 14C-2 The representative FIG. 14C-1 ARS staining microscopy images and FIG. 14C-2 relative ARS quantification of hBM-MSCs after 21 days of culture on the BTO NPs/P(VDF-TrFE) membranes. FIGS. 14D-1 and 14D-2 The representative FIG. 14D-1 immunofluorescence microscopy images and FIG. 14D-2 mean fluorescence intensities of BMP2 protein expression in hBM-MSCs after 3 days of culture on the BTO NPs/P(VDF-TrFE) membranes. (FIG. 14E, FIG. 14F) The reports on the differentiation probabilities derived from MeD-P predict the osteo-inductive function of L-phenylalanine chiral hydrogel (LH) matrices and the adipo-inductive function of D-phenylalanine chiral hydrogel (DH) matrices after 7 days of culture. FIGS. 14G-1 and 14G-2 The ALP and lipid droplet FIG. 14G-1 staining microscopy images and FIG. 14G-2 relative quantification of hBM-MSCs after 14 days of culture in the chiral hydrogels, indicating that the LH matrices significantly enhance the osteogenic lineage differentiation, and the DH matrices significantly promote the adipogenic lineage differentiation. Error bars represent standard error of mean, n=3. ** p<0.01, *** p<0.001, indicating a statistically significant difference.

FIGS. 15A and 15B shows FIG. 15A the mean differentiation index scores of hBM-MSCs after 7 days of culture on the unpolarized and polarized BTO NPs/P(VDF-TrFE) composite membranes. FIG. 15B The heat map shows that the expression of the osteogenic marker genes in hBM-MSCs after 7 days of culture on the unpolarized and polarized BTO NPs/P(VDF-TrFE) composite membranes is upregulated, and the expression of the adipogenic marker genes in hBM-MSCs after 7 days of culture on the unpolarized BTO NPs/P(VDF-TrFE) composite membranes is upregulated. Error bars represent standard error of mean, n=3.

FIGS. 16A and 16B shows FIG. 16A the mean differentiation index scores of hBM-MSCs after 7 days of incubation on the LH matrices and the DH matrices. FIG. 16B The heat map shows that in hBM-MSCs after 7 days of culture, the expression of the adipogenic marker genes in the DH matrices, the osteogenic marker genes in the LH matrices and the chondrogenic marker genes in both matrices is upregulated. Error bars represent standard error of mean, n=3.

### DETAILED DESCRIPTION OF EMBODIMENTS

The present invention will be further described with reference to the following examples.

### Example 1

The present invention provides a method for predicting and evaluating the function of a biomaterial, comprising the following steps: (1) in the environment of a material to be tested, culturing human bone marrow mesenchymal stem cells; (2) collecting the human bone marrow mesenchymal stem cells cultured in step (1), extracting total RNA from the cells, and subjecting the total RNA to purification, library construction and transcriptome sequencing; and (3) subjecting transcriptome data of samples to be tested (i.e., the data of the samples obtained in step (2)) to batch effect adjustment and feature extraction, then inputting a function prediction and evaluation model of the present invention (which is an MeD-P intelligent prediction model constructed by integrating nine machine learning algorithms of SVM-R, SVM-L, RF, GNB, LDA, LR, MLP, RidgeCV and kNN), and calculating the confidence coefficients of the samples to be tested being four cell classes of osteoblasts, chondrocytes, adipocytes and undifferentiated mesenchymal stem cells, respectively.

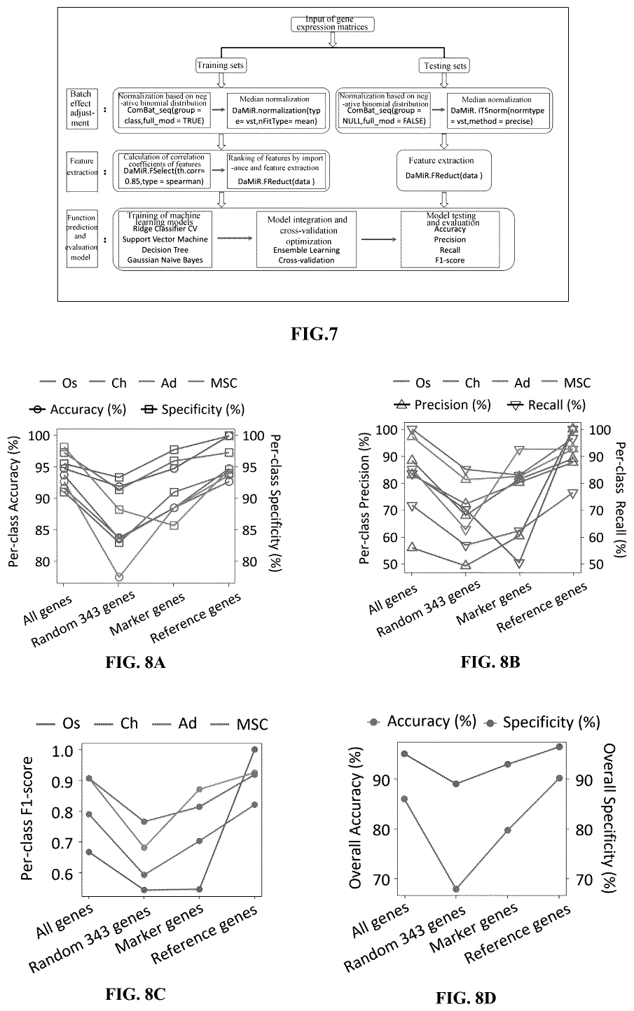

As shown in FIG. 7, the construction of the function prediction and evaluation model in the present invention comprises the following steps: firstly, dividing the transcriptome data into training sets and testing sets, and subjecting the training sets and the testing sets to batch effect adjustment, respectively; then, extracting the gene expression profiles of four cell classes based on data of the training sets, and subjecting the transcriptome data to feature extraction; afterwards, training and optimizing machine learning models based on the data of the training sets, and obtaining an MeD-P intelligent prediction model after integration; and finally, inputting data of the testing sets to the MeD-P intelligent prediction model to obtain predicted cell classes of samples in the testing sets, comparing the predicted cell classes with true cell classes of the samples, and calculating the indices of accuracy, precision, recall and F1-score of the model.

I. Batch effect adjustment: Integrated optimization based on ComBatseq algorithm and DaMiRseq algorithm.

The classes and batches of samples in the training sets were known, and the function parameters selected for their batch effect adjustment were shown in FIG. 7; and the classes of the samples in the testing sets were unknown, the batch effect adjustment of the testing sets was based on parameters generated by the batch effect adjustment of the training sets, each testing set was adjusted independently, and the function parameters selected were shown in FIG. 7.

II. Feature extraction: Integrated extraction based on DaMiRseq algorithm and DESeq2 algorithm.

After subjecting the training sets to batch effect adjustment, specifically expressed genes of the four cell classes were extracted according to the classes of the samples, and the function parameters selected were shown in FIG. 7; and then expression matrices of the feature genes were extracted from the data of the training sets and the testing sets after batch effect adjustment, respectively.

III. Function prediction and evaluation model: MeD-P intelligent prediction model constructed by integrating nine machine learning algorithms of SVM-R, SVM-L, RF, GNB, LDA, LR, MLP, RidgeCV and kNN. The model was first trained and optimized on the training sets, and then the evaluation indices of the model were calculated on the testing sets.

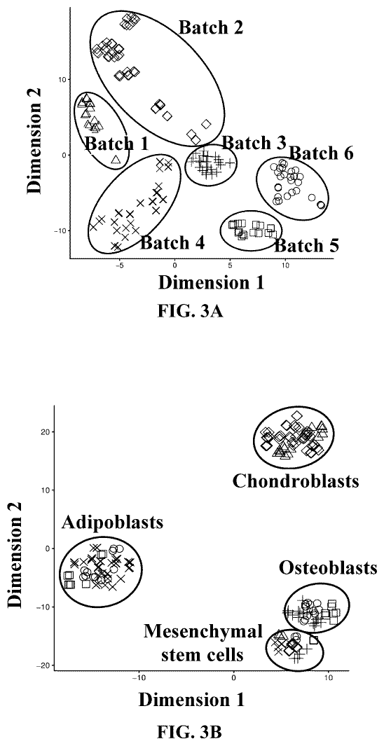

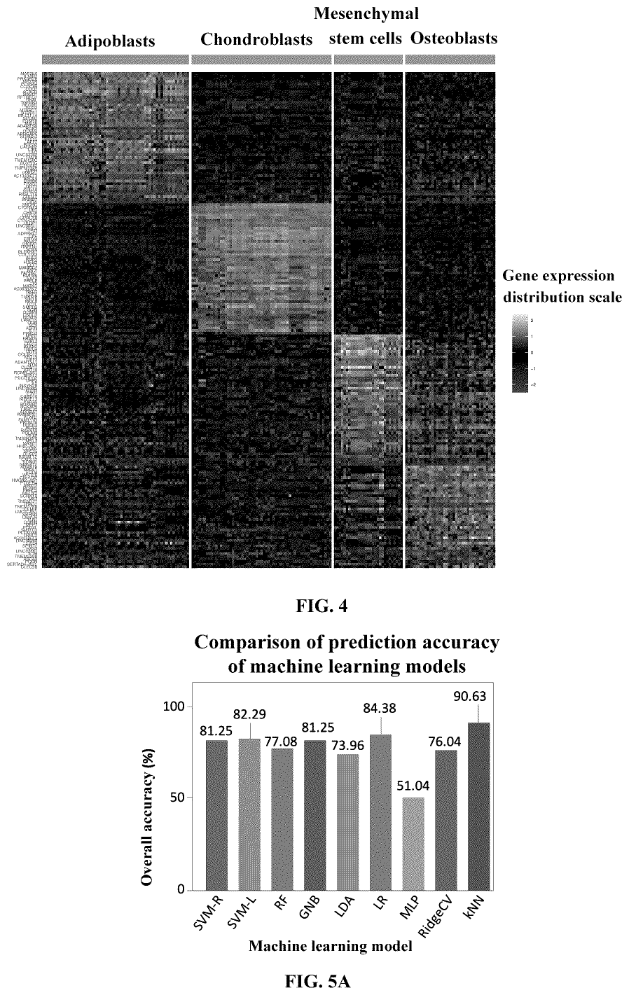

As shown in FIG. 3A, FIG. 3B and FIG. 4, in the present invention, after two data preprocessing steps of batch effect adjustment and feature extraction, there existed obvious interclass differences in the gene expression profiles of samples of four cell classes of osteoblasts, chondrocytes, adipocytes and undifferentiated mesenchymal stem cells in the reference database.

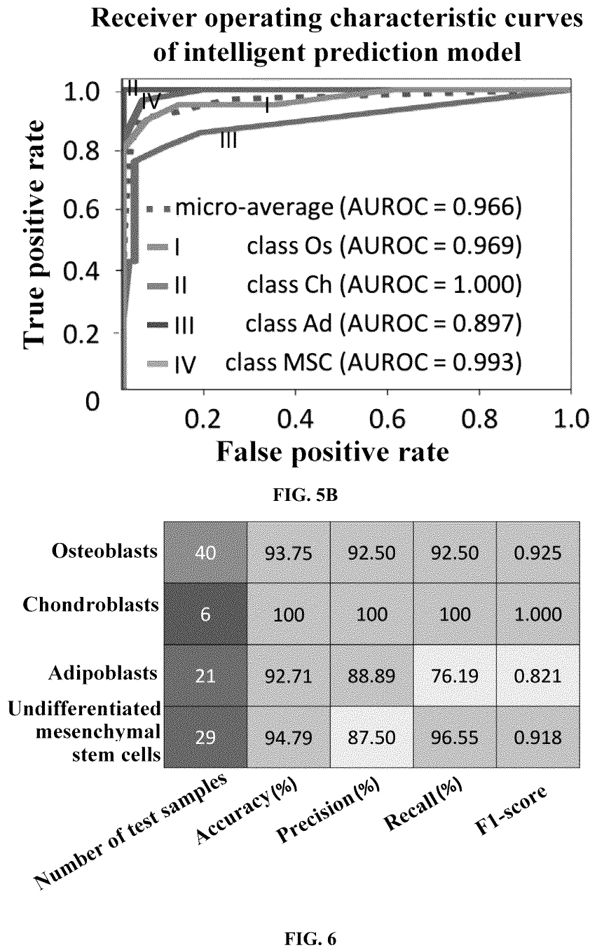

As shown in FIG. 5B, the kNN model, the default setting of the MeD-P intelligent prediction model, could distinguish samples of four cell classes of osteoblasts, chondrocytes, adipocytes and undifferentiated mesenchymal stem cells accurately. The receiver operating characteristic curves showed that the MeD-P intelligent prediction model exhibits excellent classification robustness in both the bi-classification and the multi-classification of the four cell classes.

As shown in FIG. 6, the RNAseq data derived from the public databases of human bone marrow mesenchymal stem cells before and after chemical induction to three directions of osteogenesis, chondrogenesis and adipogenesis were input to the intelligent prediction model as test samples, and the predicted cell class of each sample was obtained after calculation, so as to evaluate the classification performance of the MeD-P intelligent prediction model. It can be seen that the test samples of the four classes could all obtain a high F1-score, in which the precision and recall of the test samples of the cell class of osteoblasts were both high, indicating that the MeD-P intelligent prediction model has reliable performance in predicting whether samples cultured in the environment of a biomaterial are differentiated to osteocytes.

### Example 2

This example shows that the model trained on the selected reference feature genes outperforms those trained on the traditional marker genes and the all-gene set.

When training the machine learning models, we hypothesized that the lineage-specific genes selected based on big data were better than the known traditional lineage-specific marker genes as the training features. In order to verify this hypothesis, in this example, a kNN model based on the traditional marker genes (Table 1) was generated by training the same training samples with the same machine learning methods, and predictions were performed on the same testing datasets with the same procedures. In order to eliminate the interference of the number of genes, we also tested the models trained on the randomly-selected gene sets with the same number of genes as the selected reference genes, as well as that trained on all genes that passed the quality control. Comparisons of these models on the testing datasets showed that as compared to those trained on the traditional marker genes, the randomly-selected gene sets, and the all-gene set, the model trained on the reference feature genes selected based on big data had higher per-class accuracy, per-class specificity, per-class precision, per-class recall, and per-class F1-score (FIGS. 8A-8C and Table 2), and the highest overall accuracy and overall specificity (FIG. 8D).

TABLE 1

| Osteogenesis | | Chondrogenesis | | Adipogenesis | | Undifferentiation |
|---|---|---|---|---|---|---|
| RUNX2 | BMP2 | COL10A1 | ACAN | PPARG | ADD1 | CD44 |
| GPNMB | OPTN | COL11A1 | TGFB1 | CEBPB | CEBPZ | MASP1 |
| COL8A1 | STAT1 | COL9A2 | SOX9 | CEBPD | STAT3 | ALCAM |
| BMPR1B | ALPL | COL8A2 | SOX5 | NCOR1 | STAT2 | ENG |
| BMPR1A | CKB | COL24A1 | SOX5 | ARNTL2 | ARNTL | THY1 |
| COL1A2 | DKK1 | COL27A1 | ISLR | DDIT4 | KLF15 | TFRC |
| COL1A1 | TNC | IVS1ABP | CLU | KLF13 | NCOR2 | NT5E |
| CAMK2N1 | VDR | PMEPA1 | TCF4 | DDIT3 | LEPR | CD14 |
| SMAD3 | WNT2B | ADAMTS4 | ANKH | IGFBP6 | RASD1 | |
| SPARC | TGFBR3 | CDKN2B | SDC1 | DDIT4L | RAP2A | |
| SMAD4 | TGFBR2 | OLFML2B | IL11 | AKR1C2 | FABP3 | |
| BGN | CRYAB | MATN3 | LRRC17 | FAS | FABP5 | |
| | | FGFR3 | CMTM3 | ACSL1 | EPAS1 | |
| | | TGFB3 | ENPP1 | G0S2 | KLF2 | |
| | | AEBP1 | STAG1 | CFD | FASN | |
| | | RUNX1 | MFAP4 | SP1 | | |

TABLE 2

| | | All | Random | Marker | Reference |
|---|---|---|---|---|---|
| | | genes | 343 genes | genes | genes |
| Per-class accuracy [%] | Os | 92.70833 | 77.50577 | 88.54167 | 93.75 |
| | Ch | 94.79167 | 91.91291 | 94.79167 | 100 |
| | Ad | 91.66667 | 83.73668 | 88.54167 | 92.70833 |
| | MSC | 93.75 | 83.59916 | 88.54167 | 94.79167 |
| Per-class F1-score | Os | 0.906667 | 0.681031 | 0.870588 | 0.925 |
| | Ch | 0.666667 | 0.54313 | 0.545455 | 1 |
| | Ad | 0.789474 | 0.59244 | 0.702703 | 0.820513 |
| | MSC | 0.90625 | 0.765411 | 0.813559 | 0.918033 |
| Per-class precision [%] | Os | 97.14286 | 81.10073 | 82.22222 | 92.5 |
| | Ch | 55.55556 | 48.80086 | 60 | 100 |
| | Ad | 88.23529 | 67.64129 | 81.25 | 88.88889 |
| | MSC | 82.85734 | 71.9538 | 80 | 87.5 |
| Per-class recall [%] | Os | 85 | 62.432 | 92.5 | 92.5 |
| | Ch | 83.33333 | 69.86482 | 50 | 100 |
| | Ad | 71.42857 | 56.5117 | 61.90476 | 76.19048 |
| | MSC | 100 | 84.94492 | 82.75862 | 96.55172 |
| Per-class specificity [%] | Os | 98.21429 | 88.27275 | 85.71429 | 94.64286 |
| | Ch | 95.55556 | 93.38279 | 97.77778 | 100 |
| | Ad | 97.33333 | 91.42367 | 96 | 97.33333 |
| | MSC | 91.04478 | 83.01667 | 91.04478 | 94.02985 |
| Overall accuracy [%] | | 86.45833 | 68.40226 | 80.20833 | 90.625 |
| Overall specificity [%] | | 95.48611 | 89.46742 | 93.40278 | 96.875 |

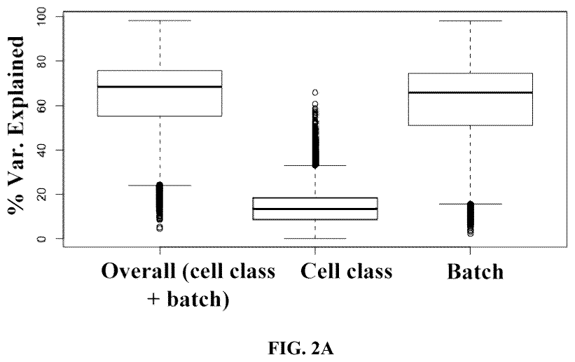

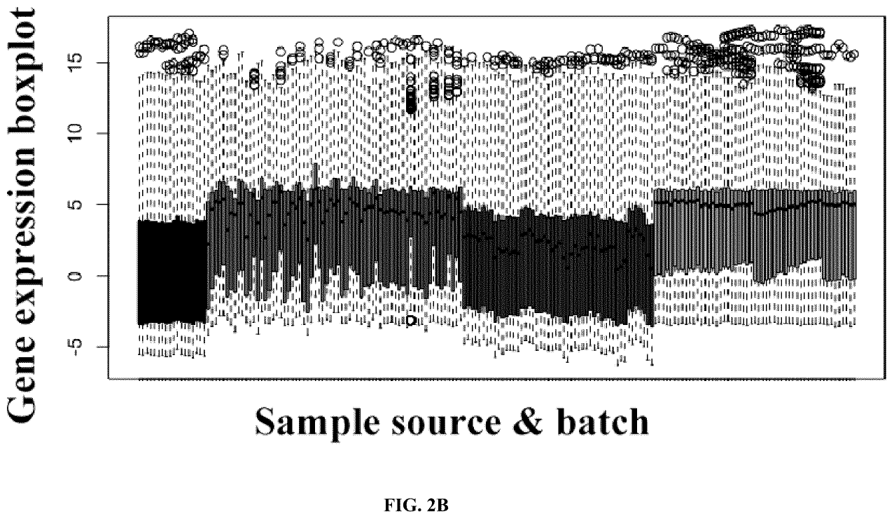

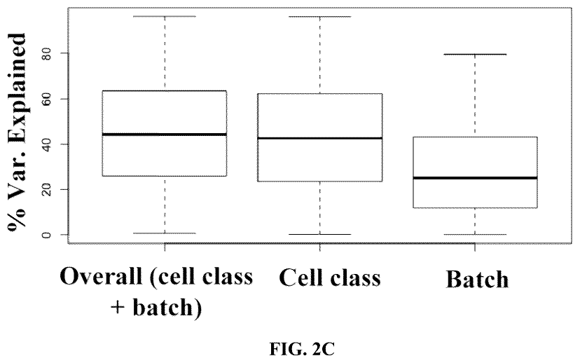

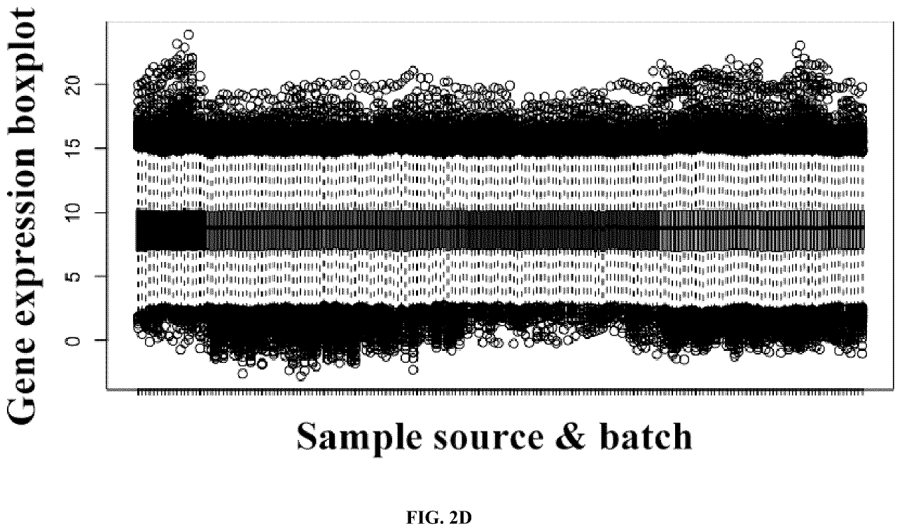

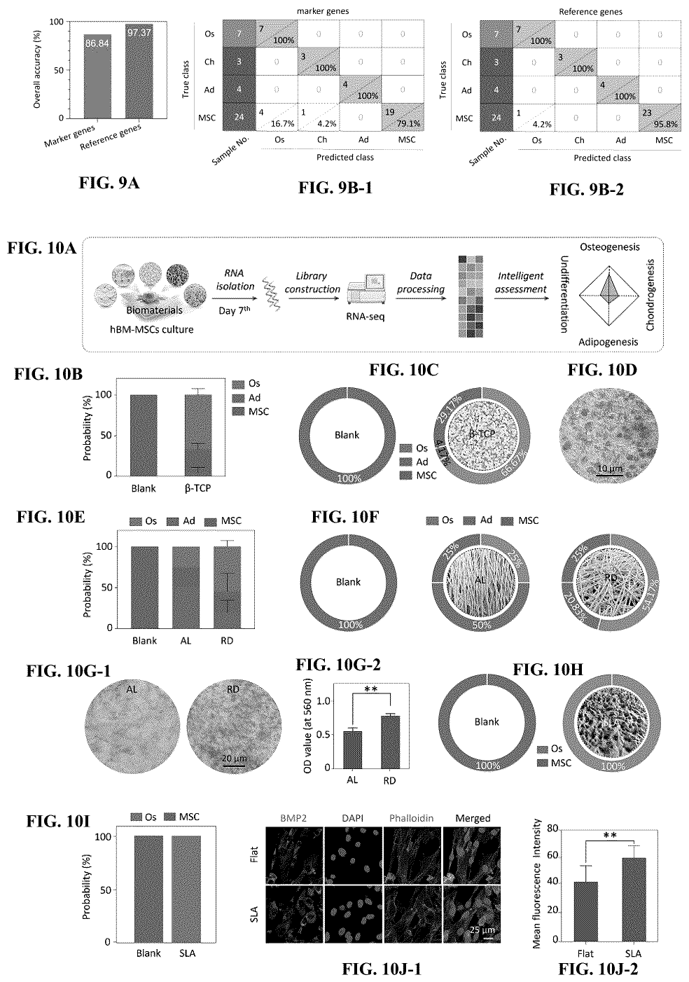

Furthermore, since Day 7 is a common timepoint for evaluating the biomaterial-induced MSCs differentiation, comparisons were also carried out between the traditional marker genes and the reference feature genes used by MeD-P using Day 7 samples from the public testing datasets and the tri-lineage differentiation experiments in our own laboratory in the present invention. The results showed that in the Day 7 sample testing sets, the reference feature genes used by MeD-P had higher overall accuracy than the traditional marker genes, especially for the undifferentiated samples (FIG. 9A-9B-2). These results thus demonstrate the potential and efficiency of the reference genes selected based on big data as the training features for the prediction model.

### Example 3

This example shows that MeD-P provides robust, accurate and rapid predictions on varied biomaterial-induced hMSCs lineage fate.

The different biomaterials used for induction were prepared as follows. For the preparation of the 3D-printed β-TCP scaffolds, see L. Chen, C. Deng, J. Li, Q. Yao, J. Chang, L. Wang, C. Wu, Biomaterials 2019, 196, 138. For the preparation of the electrospun PLLA nanofibrous membranes, see W. Liu, Y. Wei, X. Zhang, M. Xu, X. Yang, X. Deng, ACS Nano 2013, 7, 6928. For the preparation of the BTO NPs/P(VDF-TrFE) nanocomposite membranes, see X. Zhang, C. Zhang, Y. Lin, P. Hu, Y. Shen, K. Wang, S. Meng, Y. Chai, X. Dai, X. Liu, Y. Liu, X. Mo, C. Cao, S. Li, X. Deng, L. Chen, ACS Nano 2016, 10, 7279. For the preparation of the phenylalanine hydrogel matrices, see Y. Wei, S. Jiang, M. Si, X. Zhang, J. Liu, Z. Wang, C. Cao, J. Huang, H. Huang, L. Chen, S. Wang, C. Feng, X. Deng, L. Jiang, Adv. Mater. 2019, 31, 1900582. The contents of the above references are incorporated herein by reference in their entirety. The sandblasting and acid-etching (SLA)-treated Ti-6Al-4V alloy substrates were prepared as follows. White corundum with a particle size of 250-300 μm was used to sandblast the pure titanium substrates uniformly at a distance of 3-5 cm under standard atmospheric pressure. Then, the samples were ultrasonically cleaned with acetone, anhydrous ethanol and deionized water for 10 min, and then dried. The sandblasted titanium alloy substrates were placed in an acid etching solution mixed with an equal volume of 18% HCl and 48% H₂SO₄ at 60° C. for 30 min. Then, the substrates were ultrasonically cleaned with deionized water 3 times for 15 min each time, and placed in a vacuum oven at 55° C. for 12 h. Ti-6Al-4V titanium alloy substrates with polished surfaces were prepared as control.

For evaluation, human bone marrow mesenchymal stem cells (hBM-MSCs) were cultured on the representative biomaterials, and harvested after 7 days for RNA sequencing. The processed RNAseq data were loaded into MeD-P, and a report on the tri-lineage differentiation probabilities was generated (FIG. 10A).

Then, the RNA-seq data were respectively collected from hBM-MSCs after 7 days of culture on the 3D-printed β-TCP scaffolds, the electrospun PLLA nanofibrous membranes, the SLA-treated Ti-6Al-4V alloy substrates, the BTO NPs/P(VDF-TrFE) nanocomposite membranes and the chiral phenylalanine hydrogel matrices, and processed with MeD-P. hBM-MSCs were also cultured in a normal complete medium for 7 days, and the samples were collected as blank control. The MeD-P evaluation reports showed that the 3D-printed β-TCP scaffolds yielded the highest probability in inducing osteogenesis of hBM-MSCs (FIGS. 10B, 10C), which was validated by Alizarin Red S (ARS) staining on Day 21 (FIG. 10D). The differentiation index scores calculated based on the AddModuleScore function in the R package Seurat showed that hBM-MSCs cultured on the 3D-printed β-TCP scaffolds yielded higher osteogenic differentiation scores compared with those cultured on the blank control (FIG. 11A). The expression of the osteogenic marker genes such as ALPL, RUNX2 and COLIA2 in hBM-MSCs after 7 days of culture on the 3D-printed β-TCP scaffolds was upregulated, which was comparable with what was observed in the cells treated with an osteo-inductive medium (FIG. 11B).

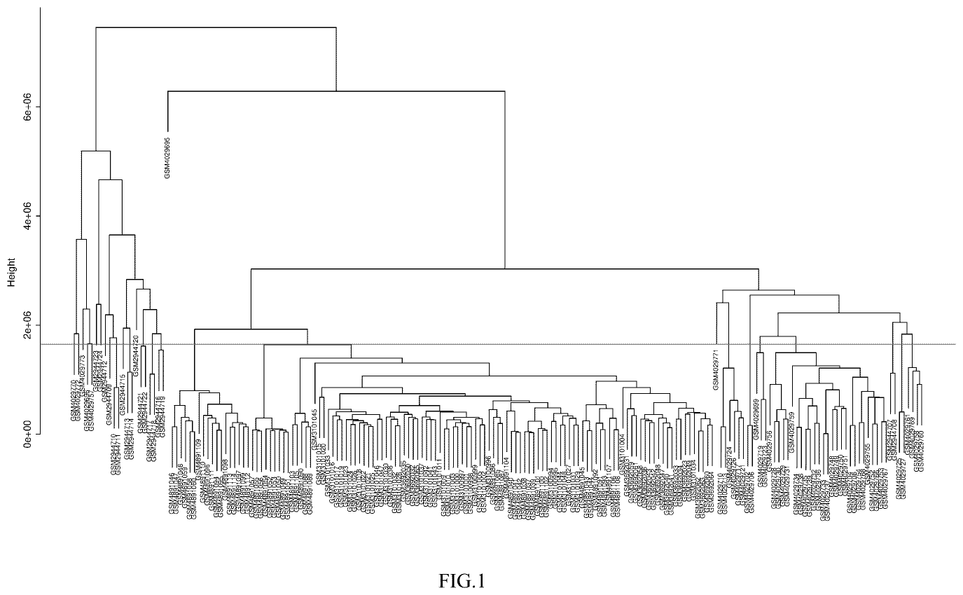

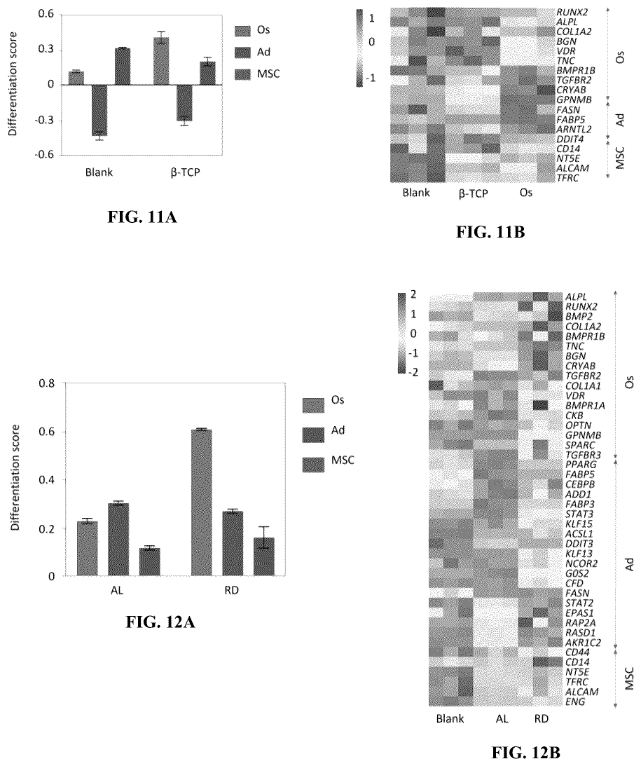

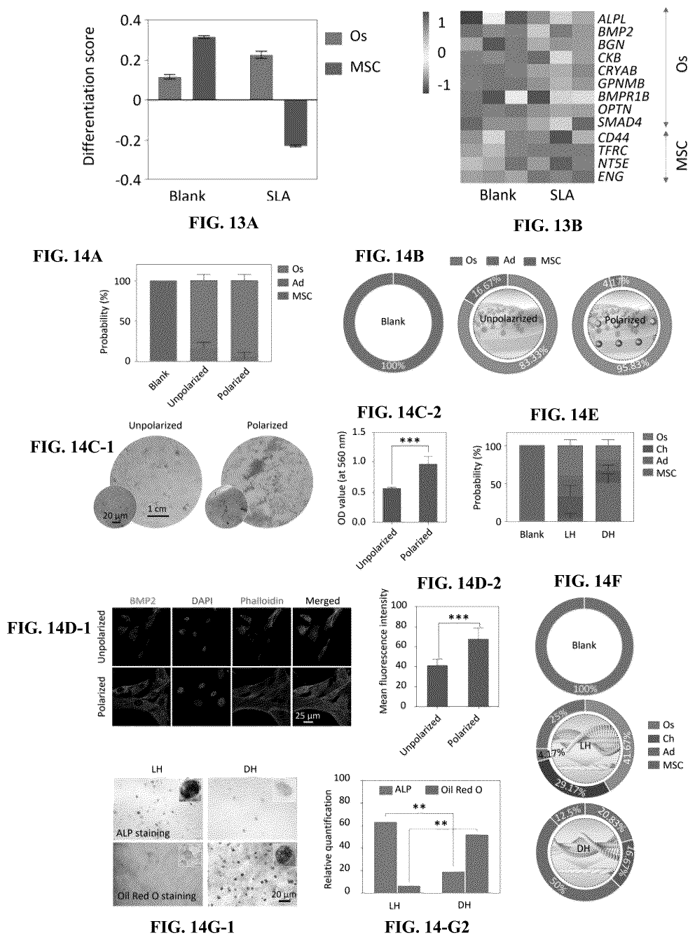

MeD-P predicted that for the electrospun PLLA nanofibrous membranes, the probability of adipogenic differentiation of hBM-MSCs cultured in the AL group (the aligned PLLA nanofibrous membranes) was the highest, and the probability of osteogenic differentiation of hBM-MSCs cultured in the RD group (the randomly-oriented PLLA nanofibrous membranes) was the highest (FIGS. 10E, 10F). The osteogenic differentiation potential of hBM-MSCs cultured in the RD group was validated by Alizarin Red S staining on Day 21 of cell culture (FIGS. 10G-1 and 10G-2). The microscopy images and relative quantification of Alizarin Red S-stained mineralized nodules showed that the RD group had stronger osteo-inductive capacity than the AL group. Additionally, hBM-MSCs cultured in the RD group also yielded much higher osteogenic differentiation scores than those cultured in the AL group (FIG. 12A). Examination of the gene expression profiles revealed that on Day 7 of cell culture, the expression level of the adipogenic marker genes was higher in the AL group, while the expression level of the osteogenic marker genes was higher in the RD group (FIG. 12B), which were consistent with the MeD-P predictions. Similarly, MeD-P predicted that the SLA-treated Ti-6Al-4V alloy substrates induced the osteogenic differentiation of hBM-MSCs with a 100% probability (FIGS. 10H, 10I), which was validated by the upregulated expression of the pro-osteogenic growth factor BMP2 on Day 3 of culture (FIGS. 10J-1 and 10J-2). The osteo-inductive capacity of the SLA-treated Ti-6Al-4V alloy substrates was also validated by its increased scores in inducing the osteogenic differentiation, as well as expression of the osteogenic marker genes such as ALPL, BMP2 and OPTN on Day 7 (FIGS. 13A and 13B).

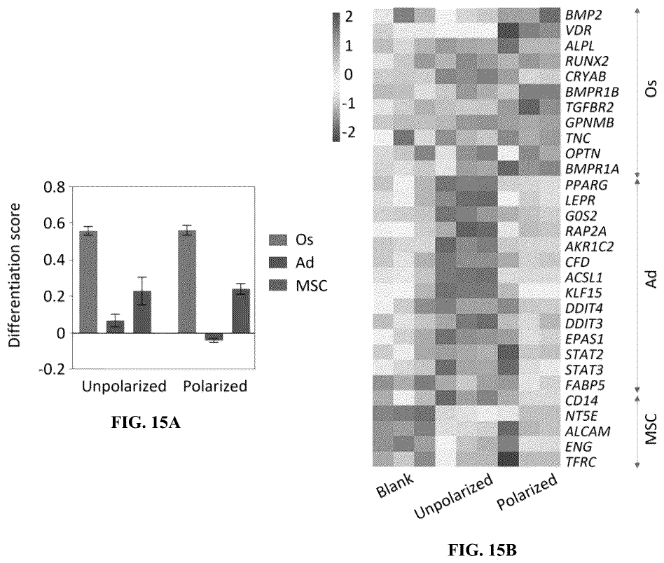

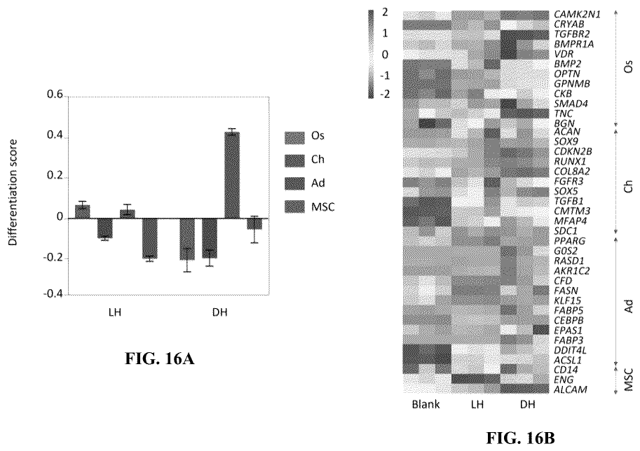

The MeD-P reports showed that the polarized BTO NPs/P(VDF-TrFE) nanocomposite membranes had significant osteogenic induction potential (FIGS. 14A, 14B), which was verified by ARS staining on Day 21 of cell culture, and the expression of the pro-osteogenic growth factor BMP2 on Day 3 of cell culture was upregulated (FIGS. 14C, 14D). It was also shown in the MeD-P prediction reports that the L-phenylalanine hydrogel could induce the osteogenic differentiation of hBM-MSCs, while the D-phenylalanine hydrogel could significantly promote adipogenesis (FIGS. 14E, 14F). The chirality-dependent lineage specification of mesenchymal stem cells was validated by alkaline phosphatase (ALP) staining and Oil Red O staining after culture of hBM-MSCs for 14 days in the chiral phenylalanine hydrogels (FIGS. 14G-1 and 14G-2), which was consistent with our previously published research results. The differentiation scores and gene expression patterns of hBM-MSCs on Day 7 of culture were consistent with the MeD-P predictions for the piezoelectric BTO NPs/P(VDF-TrFE) membranes (FIGS. 15A and 15B) and the chiral hydrogel matrices (FIGS. 16A and 16B).

However, the foregoing are only specific examples of the present invention, and cannot limit the scope of implementation of the present invention. Therefore, replacements of equivalent components of the present invention or equivalent changes and modifications made according to the scope of patent protection of the present invention should all still fall within the scope covered by the claims of the present invention.

1. A method for predicting and evaluating the function of a biomaterial, comprising the following steps:
(1) in the environment of a material to be tested, culturing human bone marrow mesenchymal stem cells;
(2) collecting the human bone marrow mesenchymal stem cells cultured in step (1), extracting total RNA from the cells, and subjecting the total RNA to purification, library construction and transcriptome sequencing to obtain transcriptome data of samples to be tested; and
(3) subjecting the transcriptome data of the samples to be tested obtained in step (2) to batch effect adjustment and feature extraction, then inputting the resulting data to a function prediction and evaluation model, and calculating the confidence coefficients of the samples to be tested being different cell classes, respectively.

2. The method for predicting and evaluating the function of a biomaterial according to claim 1, wherein the function prediction and evaluation model comprises Mesenchymal stem cell Differentiation Prediction, MeD-P.

3. The method for predicting and evaluating the function of a biomaterial according to claim 1, wherein a method for constructing the function prediction and evaluation model in step (3) comprises the following steps:
(a) dividing transcriptome data collected from the public database GEO into training sets and testing sets, and subjecting the training sets and the testing sets to batch effect adjustment, respectively;
(b) extracting the gene expression profiles of four cell classes based on data of the training sets, and subjecting the transcriptome data to feature extraction;
(c) training machine learning models based on the data of the training sets by stratified sampling at a ratio of 7:3 to obtain actual training sets and validation sets, optimizing the model based on cross-validation, selecting kNN model as the default setting, and obtaining an MeD-P intelligent prediction model after integration; and
(d) inputting data of the testing sets to the MeD-P intelligent prediction model to obtain predicted cell classes of samples in the testing sets, comparing the predicted cell classes with true cell classes of the samples, and statistically calculating the indices of accuracy, precision, recall and F1-score of the model.

4. The method for predicting and evaluating the function of a biomaterial according to claim 3, wherein the machine learning models comprise at least one of the following models: Support Vector Machine with radial basis function kernel or linear kernel (SVM-R and SVM-L), Random Forest (RF), Gaussian Naive Bayes (GNB), Linear Discriminant Analysis (LDA), Logistic Regression (LR), Multi-layer Perceptron (MLP), RidgeClassifierCV (RidgeCV) and k-Nearest Neighbor (kNN).

5. The method for predicting and evaluating the function of a biomaterial according to claim 3, wherein in step (a), the batch effect adjustment is integrated optimization based on the ComBatseq algorithm and the DaMiRseq algorithm; the classes and batches of samples in the training sets are known; and the classes of the samples in the testing sets are unknown, the batch effect adjustment of the testing sets is based on parameters generated by the batch effect adjustment of the training sets, and each testing set is adjusted independently.

6. The method for predicting and evaluating the function of a biomaterial according to claim 3, wherein in step (b), the feature extraction is integrated extraction based on the DaMiRseq algorithm and the DESeq2 algorithm; after subjecting the training sets to batch effect adjustment, specifically expressed genes of the four cell classes are extracted according to the classes of the samples; and expression matrices of the feature genes are extracted from the data of the training sets and the testing sets after batch effect adjustment, respectively.

7. The method for predicting and evaluating the function of a biomaterial according to claim 3, wherein in step (c), the model is first trained and optimized on the training sets, and then the evaluation indices of the model are calculated on the testing sets, and the MeD-P intelligent prediction model constructed comprises at least one of the following machine learning algorithm: SVM-R, SVM-L, RF, GNB, LDA, LR, MLP, RidgeCV and kNN.
